(19) United States
(12) Patent Application Publication
(10) Pub. No.: US 2005/0075567 A1
(43) Pub. Date: Apr. 7, 2005

(54) Ultrasonic diagnostic imaging system with assisted border tracing

(75) Inventors: Danny Skyba, Bothell, WA (US); Damien Dollmier, Salem, MA (US); Rohit Garg, Bothell, WA (US)

Correspondence Address: ATL ULTRASOUND, P.O. BOX 3003, 22100 BOTHELL EVERETT HIGHWAY, BOTHELL, WA, 98041-3003, US

(73) Assignee: Koninklijke Philips Electronics N.V.

(21) Appl. No.: 10/984,320

(22) Filed: Nov. 8, 2004

Related U.S. Application Data
(63) Continuation-in-part of application No. 10/025,200, filed on Dec. 18, 2001, now Pat. No. 6,692,438.
(60) Provisional application No. 60/526,574, filed on Dec. 3, 2003.

(51) Int. Cl. A61B 8/00

(52) U.S. Cl. 600/443

(57) ABSTRACT

A method and system for tracing a tissue border in a medical diagnostic image are described in which a diagnostic image containing the tissue to be traced is acquired. A user manipulates a cursor on the image display to designate three landmarks on the boundary of the tissue. An automated border detector then fits a stored boundary shape to the three landmarks. The fitted border can thereafter be adjusted to precisely fit the boundary by a rubberbanding process. In an illustrated embodiment the myocardium is traced in an image of the left ventricle by first clicking on the mitral valve corners and the apex, then fitting an endocardial border to these three landmarks, then clicking on the apex of the epicardium, then fitting an epicardial border to the epicardial apex and the mitral valve corners.

This invention claims the benefit of Provisional U.S. Patent Application Ser. No. 60/526,574, filed Dec. 3, 2003.

This is a continuation in part application of U.S. patent application Ser. No. 10/025,200, filed Dec. 18, 2001.

This invention relates to ultrasonic diagnostic imaging, and, more particularly, to a system and method for tracing the boundaries of structure and tissue in an ultrasound image.

Ultrasonic diagnostic imaging systems are capable of imaging and measuring the physiology within the body in a completely noninvasive manner. Ultrasonic waves are transmitted into the body from the surface of the skin and are reflected from tissue and cells within the body. The reflected echoes are received by an ultrasonic transducer and processed to produce an image or measurement of blood flow. Diagnosis is thereby possible with no invasion of the body of the patient.

Materials known as ultrasonic contrast agents can be introduced into the body to enhance ultrasonic diagnosis. Contrast agents are substances that strongly reflect ultrasonic waves, returning echoes which may be clearly distinguished from those returned by blood and tissue. One class of substances which has been found to be especially useful as an ultrasonic contrast agent is gases, in the form of tiny bubbles called microbubbles. Microbubbles strongly backscatter ultrasound in the body, thereby allowing tissues and blood containing the microbubbles to be readily detectable through special ultrasonic processing. Microbubble contrast agents can be used for imaging the body's vascularized tissues, such as the walls of the heart, since the contrast agent can be injected into the bloodstream and will pass through veins, arteries and capillaries with the blood supply until filtered from the blood stream in the lungs, kidneys and liver.

A diagnostic procedure which is greatly aided by contrast agents is the visualization and measurement of tissue perfusion such as the perfusion of the myocardium with oxygenated blood flow. Perfusion imaging and measurement of perfusion at a designated point in the body is described in U.S. Pat. No. 5,833,613, for instance. The parent application Ser. No. 10/025,200 describes a method and apparatus for making and displaying the results of perfusion measurements for a large region of tissue rather than just a particular sample volume location. Such a capability enables the rapid diagnosis of the perfusion rate of a significant region of tissue such as the myocardium, enabling the clinician to quickly identify small regions of tissue where perfusion is problematic due to ischemia or other bloodflow conditions.

These procedures, which perform diagnosis on a particular organ or tissue type such as the myocardium often require the precise identification of the organ or tissue being diagnosed. A technique for performing this delineation with ultrasonic images is automated or semi-automated border detection. For example, U.S. Pat. No. 6,491,636 (Chenal et al.) describes a technique for automatically tracing the endocardial border of the left ventricle of the heart which uses corner templates and septal wall angle bisection to geometrically identify the medial mitral annulus, the lateral mitral annulus and the apex of the left ventricle, then fits a border template to the three identified landmarks in the image. U.S. Pat. No. 6,346,124 (Geiser et al.) traces both the endocardial border and the epicardial border by image analysis using expert reference echocardiographic image borders. See also U.S. Pat. No. 5,797,396 (Geiser et al.) which describes a technique for identifying elliptical borders in ultrasound images.

These automated border tracing techniques, while working well with the anatomies for which they are designed, often have difficulty adapting readily to new and different organs and structures. Moreover, automated techniques are very processing-intensive and complex. Additionally, since the shapes of anatomical features can span a wide range among a population of people, automated techniques cannot be said to be foolproof. Accordingly it would be desirable to have an automated border tracing techniques which is useful with a wide variety of anatomies, is not processing intensive, and can adapt to the anatomical shapes of the majority of patients.

In accordance with the principles of the present invention an automated border tracing technique is provided which is simple to use and operate and accurate in its result. A user begins by delineating first and second landmarks on a tissue boundary of a diagnostic image. The user then delineates a third landmark on the tissue boundary and a processor then fits a border template to this first tissue boundary. The user delineates a fourth landmark on another boundary of the tissue and the processor fits a second border template to the second tissue boundary. The template shapes can then be adjusted by the user to precisely match the two tissue boundaries. In an illustrated embodiment the inventive technique is used to trace the endocardial and epicardial borders of the heart.

In the drawings:

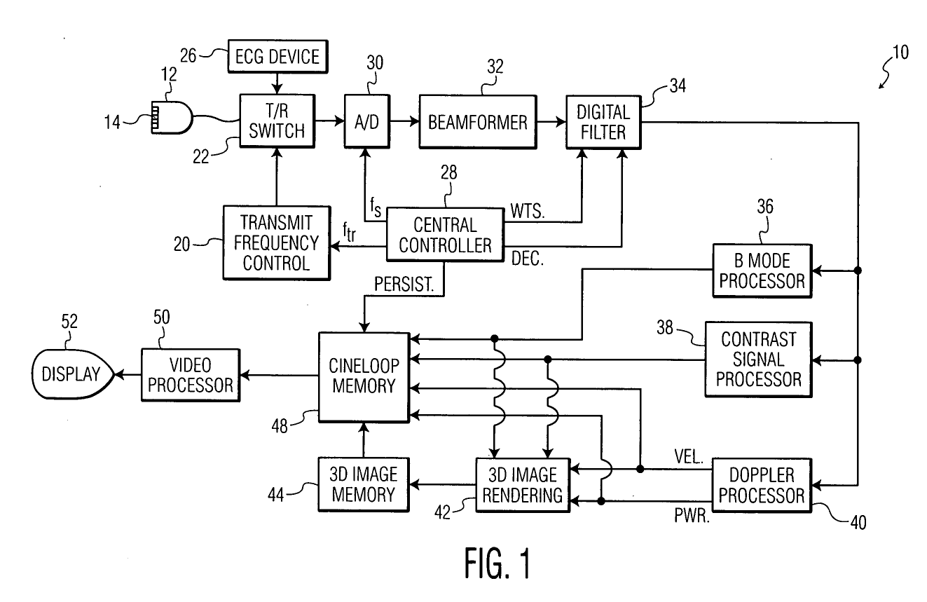

FIG. 1 is a block diagram of an ultrasonic imaging system according to one embodiment of the invention.

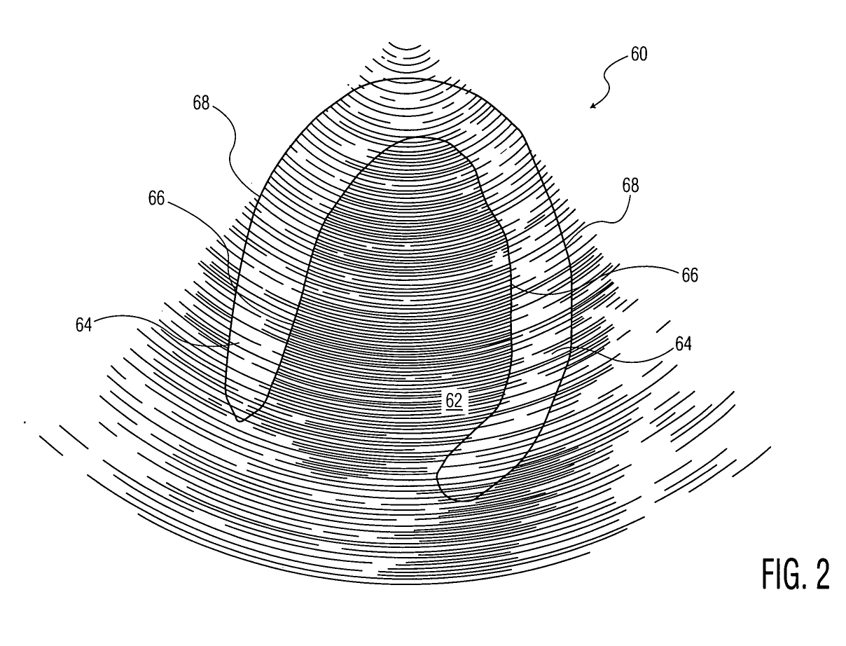

FIG. 2 is a schematic drawing showing a B-mode image of a myocardium obtained using the system of FIG. 1.

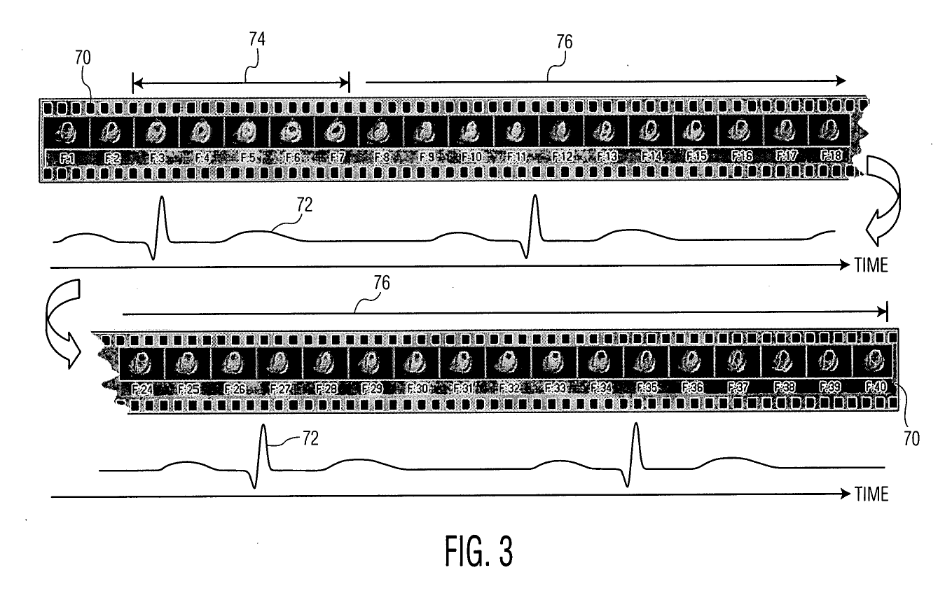

FIG. 3 illustrates the acquisition of a sequence of real time image frames for parametric imaging.

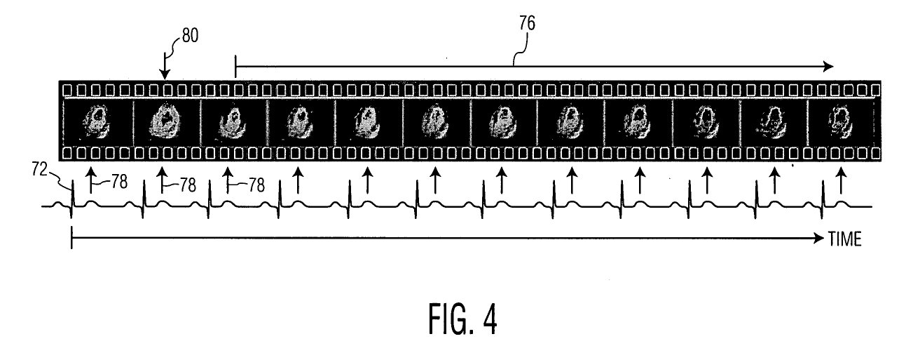

FIG. 4 illustrates gated (triggered) acquisition of a sequence of frames for parametric imaging.

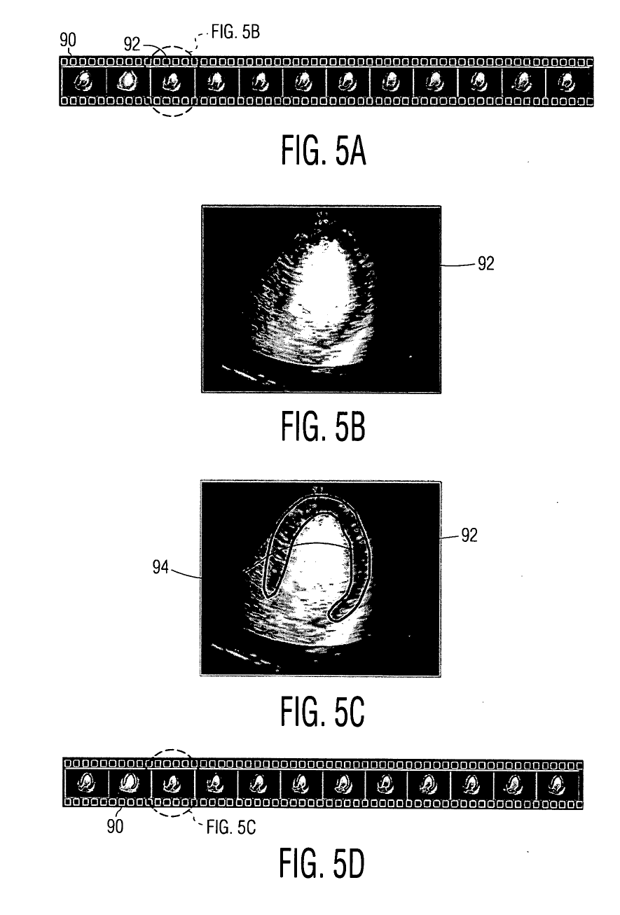

FIGS. 5a-5d illustrate the delineation of a region of interest in an image using assisted border detection.

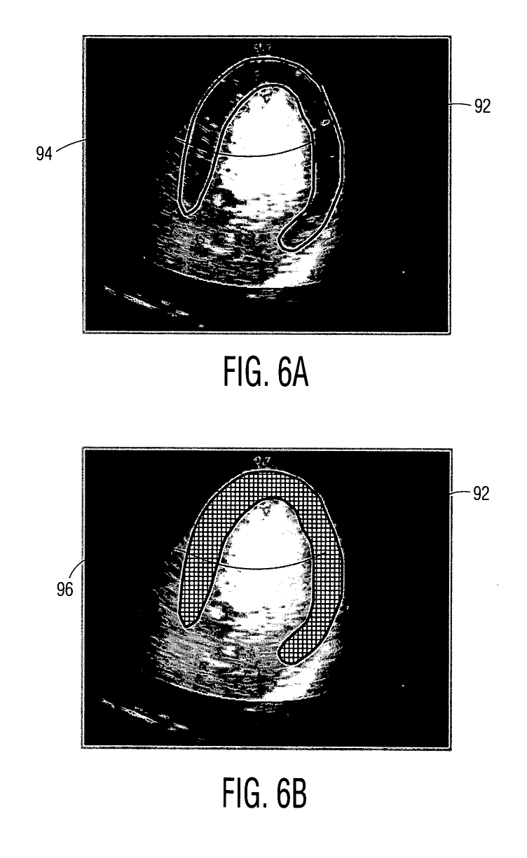

FIGS. 6a and 6b illustrate the masking of a region of interest.

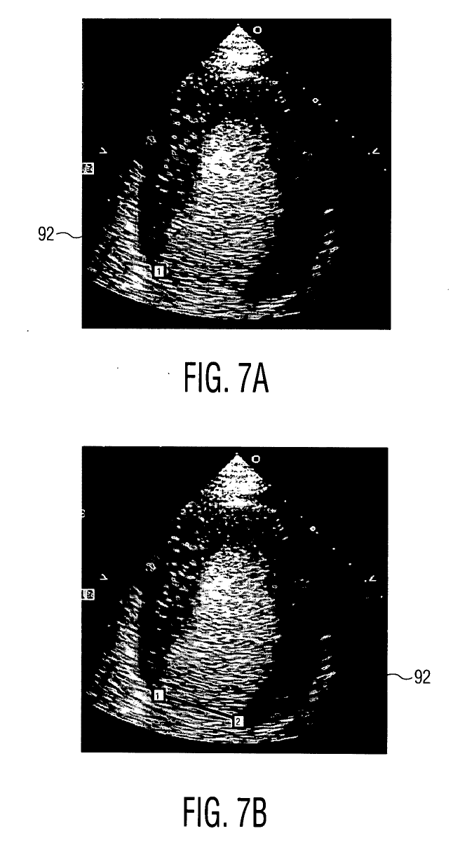

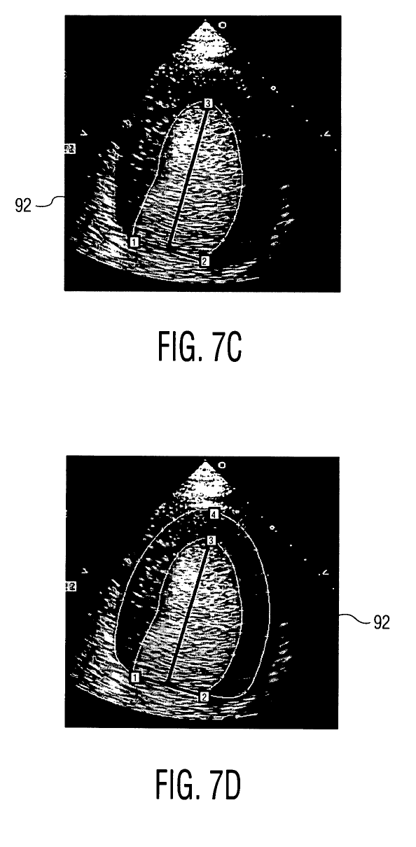

FIGS. 7a-7d are a sequence of images showing the tracing of a myocardial boundary in accordance with the principles of the present invention.

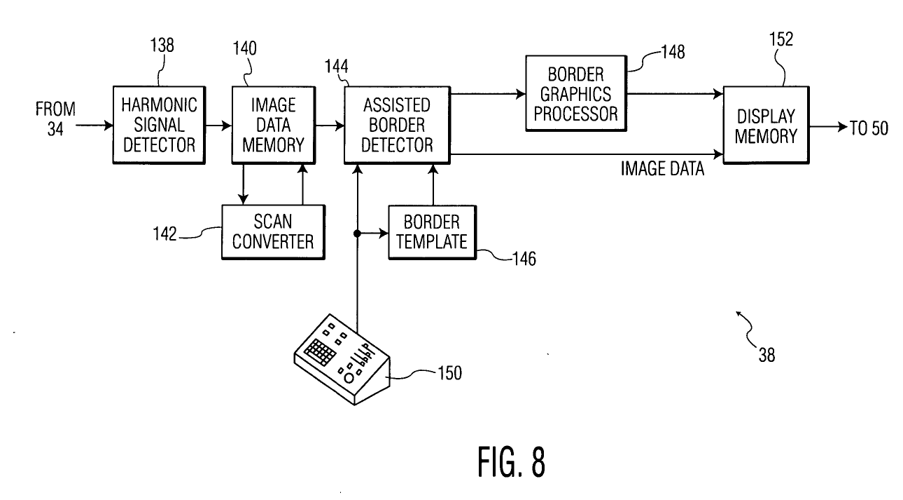

FIG. 8 illustrates in block diagram form details of an assisted border detector constructed in accordance with the principles of the present invention.

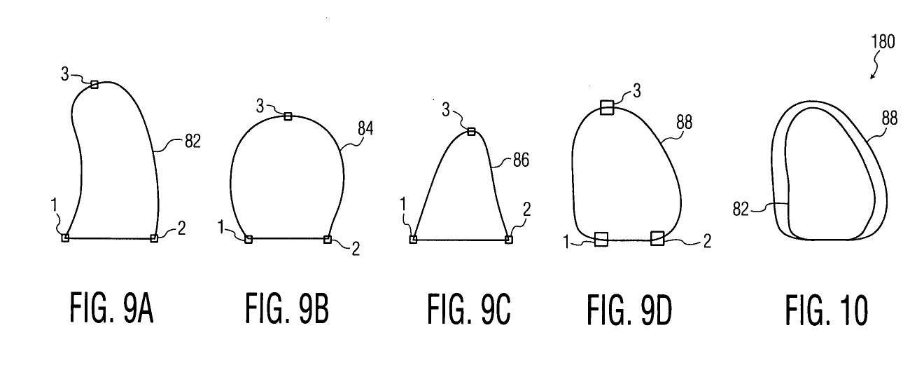

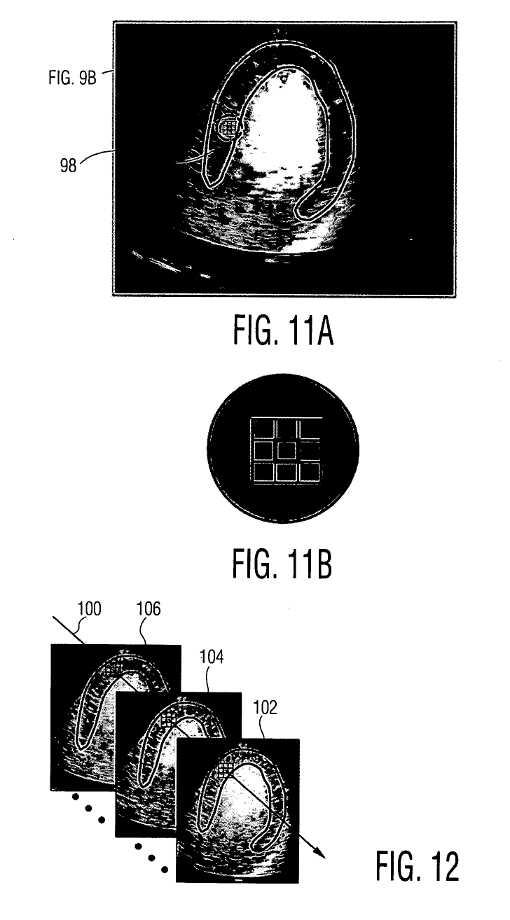

FIGS. 9a, 9b, 9c and 9d illustrate examples of stored border templates which may be utilized in an embodiment of the present invention.

FIG. 10 illustrates an epicardial border template and an endocardial border template which have been adjusted to delineate the myocardium therebetween.

FIGS. 11a and 11b illustrate a preferred technique for quantifying pixel values in a region of interest.

FIG. 12 illustrates the selection of pixel values from a plurality of images for the determination of a perfusion curve for the pixel location.

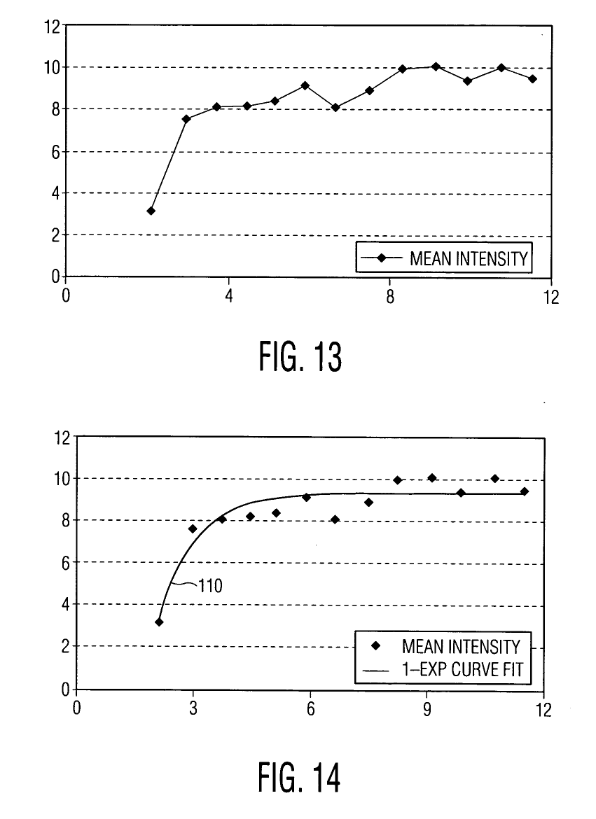

FIG. 13 illustrates the plotting of a perfusion curve from image data.

FIG. 14 illustrates the fitting of a smooth curve to the perfusion curve of FIG. 13.

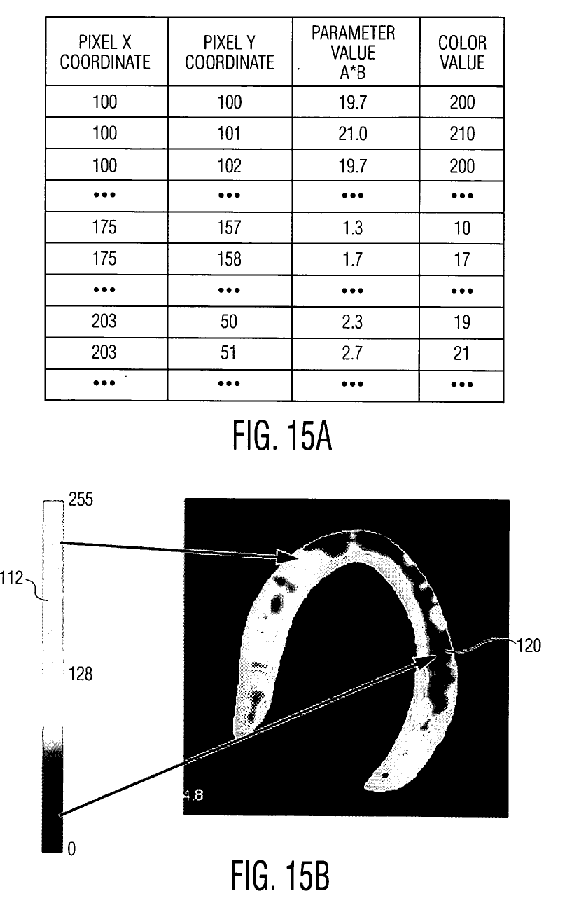

FIGS. 15a and 15b illustrate the mapping of perfusion parameters extracted from the smooth curves to a color scale and a two dimensional image.

An ultrasonic diagnostic imaging system 10 constructed in accordance with the principles of the present invention is shown in FIG. 1. An ultrasonic scanhead 12 includes an array 14 of ultrasonic transducers that transmit and receive ultrasonic pulses. The array may be a one dimensional linear or curved array for two dimensional imaging, or may be a two dimensional matrix of transducer elements for electronic beam steering in three dimensions. The ultrasonic transducers in the array 14 transmit ultrasonic energy and receive echoes returned in response to this transmission. A transmit frequency control circuit 20 controls the transmission of ultrasonic energy at a desired frequency or band of frequencies through a transmit/receive (“T/R”) switch 22 coupled to the ultrasonic transducers in the array 14. The times at which the transducer array is activated to transmit signals may be synchronized to an internal system clock (not shown), or may be synchronized to a bodily function such as the heart cycle, for which a heart cycle waveform is provided by an ECG device 26. When the heartbeat is at the desired phase of its cycle as determined by the waveform provided by ECG device 26, the scanhead is commanded to acquire an ultrasonic image. The ultrasonic energy transmitted by the scanhead 12 can be relatively high energy (high mechanical index or MI) which destroys or disrupts contrast agent in the image field, or it can be relatively low energy which enables the return of echoes from the contrast agent without substantially disrupting it. The frequency and bandwidth of the ultrasonic energy generated by the transmit frequency control circuit 20 is controlled by a control signal fₜᵣ generated by a central controller 28.

Echoes from the transmitted ultrasonic energy are received by the transducers in the array 14, which generate echo signals that are coupled through the T/R switch 22 and digitized by analog to digital (“A/D”) converters 30 when the system uses a digital beamformer. Analog beamformers may also be used. The A/D converters 30 sample the received echo signals at a sampling frequency controlled by a signal f_{S} generated by the central controller 28. The desired sampling rate dictated by sampling theory is at least twice the highest frequency of the received passband, and might be on the order of at least 30-40 MHz. Sampling rates higher than the minimum requirement are also desirable.

The echo signal samples from the individual transducers in the array 14 are delayed and summed by a beamformer 32 to form coherent echo signals. The digital coherent echo signals are then filtered by a digital filter 34. In this embodiment, the transmit frequency and the receiver frequency are individually controlled so that the beamformer 32 is free to receive a band of frequencies which is different from that of the transmitted band. The digital filter 34 bandpass filters the signals, and can also shift the frequency band to a lower or baseband frequency range. The digital filter could be a filter of the type disclosed in U.S. Pat. No. 5,833,613.

Filtered echo signals from tissue are coupled from the digital filter 34 to a B mode processor 36 for conventional B mode processing. The B mode image may also be created from microbubble echoes returning in response to nondestructive ultrasonic imaging pulses. As discussed above, pulses of low amplitude, high frequency, and short burst duration will generally not destroy the microbubbles.

Filtered echo signals of a contrast agent, such as microbubbles, are coupled to a contrast signal processor 38. The contrast signal processor 38 preferably separates echoes returned from harmonic contrast agents by the pulse inversion technique, in which echoes resulting from the transmission of multiple pulses to an image location are combined to cancel fundamental signal components and enhance harmonic components. A preferred pulse inversion technique is described in U.S. Pat. No. 6,186,950, for instance, which is hereby incorporated by reference. The detection and imaging of harmonic contrast signals at low MI is described in U.S. Pat. No. 6,171,246, the contents of which is also incorporated herein by reference.

The filtered echo signals from the digital filter 34 are also coupled to a Doppler processor 40 for conventional Doppler processing to produce velocity and power Doppler signals. The outputs of these processors may be displayed as planar images, and are also coupled to a 3D image rendering processor 42 for the rendering of three dimensional images, which are stored in a 3D image memory 44. Three dimensional rendering may be performed as described in U.S. Pat. No. 5,720,291, and in U.S. Pat. Nos. 5,474,073 and 5,485,842, all of which are incorporated herein by reference.

The signals from the contrast signal processor 38, the processors 36 and 40, and the three dimensional image signals from the 3D image memory 44 are coupled to a Cineloop® memory 48, which stores image data for each of a large number of ultrasonic images. The image data are preferably stored in the Cineloop memory 48 in sets, with each set of image data corresponding to an image obtained at a respective time. The sets of image data for images obtained at the same time during each of a plurality of heartbeats are preferably stored in the Cineloop memory 48 in the same way. The image data in a group can be used to display a parametric image showing tissue perfusion at a respective time during the heartbeat. The groups of image data stored in the Cineloop memory 48 are coupled to a video processor 50, which generates corresponding video signals for presentation on a display 52. The video processor 50 preferably includes persistence processing, whereby momentary intensity peaks of detected contrast agents can be sustained in the image, such as described in U.S. Pat. No. 5,215,094, which is also incorporated herein by reference.

The manner in which perfusion can be displayed in a parametric image will now be explained beginning with reference to FIG. 2. An ultrasound image 60 is obtained from a region of interest, preferably with the aid of microbubbles used as a contrast agent, as shown in FIG. 2. The anatomy shown in FIG. 2 is the left ventricle 62 of a heart, although it will be understood that the region of interest can encompass other tissues or organs. The left ventricle 62 is surrounded by the myocardium 64, which has inner and outer borders, 66, 68, respectively, that defines an area of interest, the perfused myocardium 64. The myocardium can be distinguished for analysis by segmentation either manually or automatically using conventional or hereinafter developed techniques, as described below.

FIG. 3 illustrates a real time sequence 70 of images of the myocardium which have been acquired with a contrast agent present in the heart. The image frames in the sequence are numbered F:1, F:2, F:3, and so on. The sequence is shown in time correspondence to an ECG waveform 72 of the heart cycle. It will be appreciated that during a heart cycle 10, 20, 30, 40 or more images may be acquired, depending upon the heart rate and the ultrasound system frame rate. In one embodiment of the present invention the acquired sequence 70 of images is stored in the Cineloop memory 48. In this embodiment, during one interval 74 of images, high MI pulses are used to acquire the images. This is typically an interval of 1-10 image frames. The use of the high intensity transmit pulses substantially disrupts or destroys the microbubbles in the image plane or volume. In this discussion these high MI frames are referred to as “flash” frames. At the end of this interval 74 low MI pulses are used to image subsequent image frames over several cardiac cycles delineated by interval 76 as the contrast agent re-perfuses the myocardium. The sequence of images shows the dynamics of the cardiac cycle as well as contrast replenishment over many heart cycles.

Instead of acquiring a continual real time sequence of images, images can be selected out of a real time sequence or acquired at specific times in the cardiac cycle. FIG. 4 illustrates this triggered acquisition, in which the arrows 78 indicate times triggered from the ECG waveform 72 at which images are acquired at a specific phase of the heart cycle. The arrow 80 indicates the time when one or more flash frames are transmitted, followed by an interval 76 during which low MI images are acquired. In this example only one image is acquired and stored in Cineloop memory during each cardiac cycle. The user sets the trigger timing to determine which phase of the cardiac cycle to capture with the triggered images. When these images are replayed from Cineloop memory in real time, they do not show the dynamics of the cardiac cycle, as the heart is at the same phase of the cardiac cycle during each image. The sequence does show contrast replenishment in the triggered images acquired during the low MI interval 76. From image to image the viewer can see the buildup of blood in the myocardial tissue as each beat of the heart sends more blood with microbubbles into the myocardial tissue. From a time immediately following the flash frame re-perfusion can be visually observed as the myocardium becomes brighter with more microbubbles infused with each heartbeat. Tissue which does not light up as rapidly as, or to a lesser final level than, neighboring tissue can indicate the possibility of a pathological condition such as an arterial obstruction or other defect.

The region of interest in an image, in this example the myocardium, may be delineated by assisted border detection as shown in FIGS. 5a-5d. FIG. 5a illustrates a contrast image sequence 90 which may be a real time sequence 70 or a triggered sequence 80. From the image sequence 90 the user selects an image 92 which shows relatively well defined endocardial and epicardial borders. This image 92 is shown enlarged in FIG. 5b. The selected image may then processed by assisted border detection, as described in U.S. Pat. No. 6,491,636, entitled “Automated Border Detection in Ultrasonic Diagnostic Images,” the contents of which is hereby incorporated by reference. Automated or assisted border detection acts to delineate the myocardium with a border 94 as shown in FIGS. 5c and 6a. The border outline 94 on the selected image is then used to automatically delineate the border on other images in the sequence 90, as explained in the '636 patent and shown in FIG. 5d. Alternatively, the borders may be drawn on the other images in the sequence by processing them individually with the automated border detection algorithm. The region of interest where perfusion is to be represented parametrically is now clearly defined for subsequent processing. If desired, the area of interest may be further defined by a mask 96, as shown in FIG. 6b, in which the area within the border trace is masked. All pixels under the mask are to be processed in this example, while pixels outside of the mask are not processed parametrically.

In accordance with the principles of the present invention, the myocardium of the left ventricle is delineated by an assisted border detection technique as follows. The user displays an image 92 on which the border is to be traced as shown in FIG. 7a. The user designates a first landmark in the image with a pointing device such as a mouse or a trackball usually located on the system control panel which manipulates a cursor over the image. In the example of FIG. 7a, the first landmark designated is the medial mitral annulus (MMA). When the user clicks on the MMA in the image, a graphic marker appears such as the white control point indicated by the number “1” in the drawing. The user then designates a second landmark, in this example the lateral mitral annulus (LMA), which is marked with the second white control point indicated by the number “2” in FIG. 7b. A line then automatically connects the two control points, which in the case of this longitudinal view of the left ventricle indicates the mitral valve plane. The user then moves the pointer to the endocardial apex, which is the uppermost point within the left ventricular cavity. As the user moves the pointer to this third landmark in the image, a template shape of the left ventricular endocardial cavity dynamically follows the cursor, distorting and stretching as the pointer seeks the apex of the chamber. This template, shown as a white line in FIG. 7c, is anchored by the first and second control points 1 and 2 and passes through the third control point, which is positioned at the apex when the user clicks the pointer at the apex, leaving the third control point 3. When positioned, the endocardial cavity template provides an approximate tracing of the endocardium as shown in FIG. 7c. In the embodiment of FIG. 7c a black line which bisects the left ventricle follows the pointer as it approaches and designates the apex. This black line is anchored between the center of the line indicating the mitral valve plane and the left ventricular apex, essentially indicating a center line between the center of the mitral valve and the apex of the cavity.

With the endocardial border thus defined, the user moves the cursor to the epicardial apex, the uppermost point on the outer surface of the myocardium. The user then clicks on the epicardial apex and a fourth control point marked “4” is positioned. A second template then automatically appears which approximately delineates the epicardial border as shown in FIG. 7d. This second template, shown by the outer white border line in FIG. 7d, is also anchored by the first and second control points and passes through the positioned fourth control point at the epicardial apex. The two templates are an approximate outline of the myocardial border.

As a final step, the user may want to adjust the templates shown in FIG. 7d so that they precisely outline the border of the myocardium. Located around each tracing are a number of small control points shown in the drawing as “+” symbols. The number and spacing of these small control points is a design choice or may be a variable that the user can set. The user can point at or near these control points and click and drag the outline to more precisely delineate the myocardial boundary. This process of stretching or dragging the border is known as “rubberbanding”, and is described more fully in the aforementioned '636 patent, with particular reference to FIG. 9 of that patent. As an alternative to rubberband adjustment, in a more complex embodiment the approximated borders may automatically adjust to the image borders by image processing which uses the intensity information of the pixels at and around the approximated tissue borders. When finished the border can precisely delineate the boundary of the myocardium thereby enclosing the image pixels of the region of interest needed for parametric imaging of myocardial perfusion.

Details of a contrast signal processor for performing assisted border detection as described above are shown in FIG. 8. Echo signals are received by a harmonic signal detector 138 which separates and detects harmonic signal components from echo signals returned by tissue and/or contrast agent in the blood flow. Harmonic signal separation can be performed by bandpass filtering or by pulse inversion as described in U.S. Pat. Nos. 5,706,819 (Hwang), 5,951,478 (Hwang et al.), and 6,193,662 (Hwang). The harmonic signals are detected by amplitude detection or Doppler processing (see U.S. Pat. No. 6,095,980) and stored in an image data memory 140. The image data used for an image is forwarded to a scan converter 142 which produces image data of the desired image format, e.g., sector, rectangular, virtual apex, or curved linear. The scan converted image data is stored in the image data memory from which it is accessed by an assisted border detector 144. The assisted border detector 144 is responsive to input from the trackball pointing device on a user control panel 150 to locate the control points with reference to the image data and position and stretch the boundary templates with respect to the image data. The template data is provided by a border template storage device 146. As the control points and borders are being drawn and positioned on the image, the control point and border data produced by the assisted border detector 144 is applied to a border graphics processor 148, which produces a graphic overlay of the control points and border to be displayed with the image data. The graphic overlay and the image data are stored in a display memory 152, from which they are accessed for display by the video processor 50.

Examples of the templates which are stored by the border template storage device 146 are shown in FIGS. 9a-9d. FIGS. 9a-9c are examples of endocardial border templates and illustrate three general endocardial shapes which may be represented. The template 82 of FIG. 9a is horseshoe-shaped and is generally selected by clinicians for the majority of cases. FIG. 9b shows a more circular, bulbous template 84 which may be selected for some cases and FIG. 9c shows a more pyramidal or triangular template 86 which may be selected for other cases. The user can select a desired template after acquiring an image to be traced, at which time the user can visually see the general shape of the patient's endocardium and therefore can choose the appropriate template. FIG. 9d is an example of a template 88 for the epicardial border of the left ventricle. FIG. 10 illustrates a myocardial border overlay 180 which is a combination of the template 82 for the endocardium and the template 88 for the epicardium. The endocardial and epicardial templates in a constructed embodiment can have different shapes which are tailored to the echocardiographic views which can be traced. For example, there may be different template shapes for apical 4-chamber views, apical 2-chamber views, short axis views, parasternal views, and so forth. When the assisted border detection technique of the present invention is used to delineate organs, tissues and structures other than the left ventricle, such as the fetal head and limbs or vessel walls, templates of other appropriate shapes will be used.

It is seen that the assisted border detector embodiment described above operates by fitting border templates to three landmarks placed on the tissue boundary by the user. The first three landmarks enable automatic placement of an endocardial border template and the fourth landmark is used in combination with the first two landmarks to enable automatic placement of an epicardial border template. Together the two outlined borders define the myocardium in the image.

FIGS. 11a and 11b illustrate a preferred technique for processing the pixels within a region of interest. As FIGS. 11a and 11b show, for each pixel within the region of interest a mean image intensity value is calculated for a pixel and its surrounding eight neighboring pixels. Pixel values are calculated in this manner for each pixel in the myocardium 98 in this example, and the process is repeated for every pixel in the same location for each image in the sequence as shown for images 102, 104, 106 in FIG. 12. The common location pixel values are, at least conceptually, then plotted graphically as a function of time and mean intensity as shown in FIG. 13, which shows a plot of the common location pixel values intersected by arrow 100 in FIG. 12. The common location pixels are then used to develop a perfusion parameter for display in a two- or three-dimensional image of the region of interest. In a preferred embodiment, parameters are produced by fitting the plotted values to a curve 110 of the form:
I(t)=A(1−exp^{(-B*1})+C
where A is the final curve intensity, B is proportional to the initial slope of the curve, and C is a floating constant. A drawn curve 110 of this form is illustrated in FIG. 14. Parameters may then be formed using the values. A, B, and combinations thereof (A*B, A/B, etc.) as shown below.

FIGS. 15a-15b illustrate the creation of a parametric image from a parameter value of the form A*B using the curve characteristics described above. In the table of FIG. 15a, the first two columns indicate the locational coordinates of pixels in a two dimensional image. For three dimensional images a third coordinate will be used. The A*B parameter value for each pixel location is represented in the third column. The range of parameter values, represented by the color bar 112 calibrated from zero to 255 between FIGS. 15a and 15b, is then used to encode (map) each parameter value to a color, brightness, or other display characteristic. The colors are then displayed in their respective locations in a two or three dimensional parametric image 120, as shown in FIG. 15b, in which the perfusion of the myocardium of the heart is parametrically displayed. The techniques of the present invention may be used to produce a single static image 120 as shown in FIG. 15b, or they may be used to produce a sequence of parametric images which may be displayed in sequence or in real time, as discussed more fully in the parent application Ser. No. 10/025,200.

1. A method of delineating the boundary of tissue or structure in a medical diagnostic image comprising:
acquiring an image containing tissue or structure which is to be delineated;
manually marking at least three points of the boundary which is to be delineated; and
automatically fitting a predetermined border shape to the three points of the boundary, whereby the fitted border shape indicates a boundary of the tissue or structure in the image.

2. The method of claim 1, wherein manually marking and automatically fitting further comprise:
manually marking two points of the boundary;
manipulating a cursor to move to a third point of the boundary; and
automatically fitting the predetermined border shape to the two marked points and the cursor as the cursor is moved to the third point.

3. The method of claim 1, further comprising:
automatically aligning the fitted predetermined border shape to the boundary of the tissue or structure in the image.

4. The method of claim 1, further comprising:
manually marking at least one point of a second boundary which is to be delineated; and
automatically fitting a predetermined border shape to the point of the second boundary and at least one point of the points of the first-named boundary.

5. The method of claim 4, wherein automatically fitting a predetermined border shape to the point of the second boundary comprises automatically fitting a second predetermined border shape to the point of the second boundary and two of the points of the first boundary.

6. The method of claim 1, further comprising:
manually adjusting the fitted border shape to align with the boundary of the tissue or structure in the image.

7. The method of claim 6, wherein the act of manually adjusting the fitted border shape comprises adjusting the fitted border shape by a rubberbanding adjustment.

8. The method of claim 1, wherein acquiring further comprises acquiring an ultrasonic image of the heart; and
wherein manually marking further comprises manually marking at least three points of a wall of the heart in the image,
wherein the fitted border shape indicates the heart wall in the image.

9. A method of delineating the myocardium in a cardiac image comprising:
acquiring a diagnostic image of the heart including the myocardium;
manually marking at least three points of the endocardium; and
automatically fitting a predetermined endocardial border shape to the three points of the endocardium, whereby the fitted border shape indicates a boundary of the myocardium.

10. The method of claim 9, further comprising selecting one of a plurality of predetermined endocardial border shapes to be fitted to the three points of the endocardium.

11. The method of claim 9, wherein acquiring further comprises acquiring an echocardiographic image of the left ventricle;
wherein manually marking further comprises manually marking three landmarks on the endocardium in the image of the left ventricle; and
wherein automatically fitting further comprises automatically fitting a predetermined left ventricle endocardial border shape to the three landmarks.

12. The method of claim 11, wherein manually marking three landmarks further comprises marking the MMA, the LMA and the apex of the left ventricle in the image.

13. The method of claim 9, further comprising manually marking a point of the epicardium; and
automatically fitting a predetermined epicardial border shape to the point of the epicardium and at least one point of the endocardium.

14. An ultrasonic diagnostic imaging system for delineating an anatomical boundary in an image comprising:
a scanhead having an array transducer for scanning a region of interest;
a beamformer coupled to the array transducer which acts to beamform echo signals received from the region of interest;
an image processor coupled to the beamformer which acts to form an image of the region of interest;
a user operated pointing device which enables a user to manipulate a cursor in the image and to identify at least three points on an anatomical boundary in the image;
a source of border shapes; and
an assisted border detector, coupled to the source of border shapes and responsive to the image processor and the pointing device which acts to fit the border shapes to the points identified by the user operated pointing device.

15. The ultrasonic diagnostic imaging system of claim 14 further comprising:
a graphics processor, responsive to the border shape fitted by the assisted border detector, which acts to produce a graphic overlay including the fitted border shape; and
an image display responsive to the image processor and the graphics processor for producing an image of the region of interest with a delineated boundary.

16. The ultrasonic diagnostic imaging system of claim 15, further comprising a selector, coupled to the source of border shapes, which enables selection of one of the border shapes for use by the assisted border detector.

17. The ultrasonic diagnostic imaging system of claim 14, wherein the scanhead further comprises a scanhead having an array transducer for scanning a volumetric region of interest.
